(19) United States
(12) Patent Application Publication
(10) Pub. No.: US 2007/0015226 A1
(43) Pub. Date: Jan. 18, 2007

(54) Method for detecting cancer using metal-oxide or metal-sulfide nanoparticle fluorescent material

(75) Inventors: Hiroyuki Hirai, Ashigarakami-gun (JP); Masayoshi Kojima, Ashigarakami-gun (JP); Junji Nishigaki, Ashigarakami-gun (JP)

Correspondence Address: BUCHANAN, INGERSOLL & ROONEY PC, POST OFFICE BOX 1404, ALEXANDRIA, VA, 22313-1404, US

(73) Assignee: FUJI PHOTO FILM CO., LTD., Minami-ashigara-shi (JP)

(21) Appl. No.: 11/483,103

(22) Filed: Jul. 10, 2006

(30) Foreign Application Priority Data
Jul. 12, 2005 (JP) 2005-202467

(51) Int. Cl. G01N 33/574; C07K 16/46

(52) U.S. Cl. 435/7.23; 530/388.8; 530/391.1; 977/900

(57) ABSTRACT

It is an object of the present invention to provide a nanoparticle fluorescent material for detecting cancer with which cancer can be detected with high sensitivity, using highly safe and broad light emission on a simple device. The present invention provides a nanoparticle fluorescent material which comprises a metal-oxide or metal-sulfide nanoparticle fluorescent material whose surface is modified by a surface modifying agent and whose half bandwidth of light emission is between 50 and 200 nm, wherein an antibody that recognizes cancer antigen is bound to the surface modifying agent.

### TECHNICAL FIELD

The present invention relates to a metal-oxide or metal-sulfide nanoparticle fluorescent material which is used for detecting cancer, and to a method for detecting cancer using the nanoparticle fluorescent material.

### BACKGROUND ART

It is known that particulate materials of nanometer size exhibit properties different from those of bulk materials. For example, in the case of semiconductors, the so-called quantum size effect is well known, in which the band gap, which has been believed to be material-specific, varies depending on the particle size. The particle size at which this effect becomes significant differs depending on the type of semiconductor material; however, it is generally from several dozens of nm to a few nm. Thus, single nanoparticle is particularly important. There are also materials for which an effect is known, whereby, as the quantum size effect becomes significant, the life of fluorescence becomes short and light emission, which would otherwise not be observed, can be observed, for example. As stated above, since nano-sized materials, especially single-nano-sized materials, have properties different from those of conventionally known bulk materials, they are drawing much attention in science and engineering.

For example, there is proposed a semiconductor nanoparticle fluorescent material for detecting a target molecule by binding a molecular probe onto the surface of beads which were prepared from semiconductor nanoparticles such as CdSe/CdS (core/shell) or CdSe/ZnS (core/shell). These semiconductor nanoparticles can be caused to emit light at different wavelengths by varying their crystallite sizes. It is also possible to carry out simultaneous multiple measurements by encoding labeled beads with a combination of light emission wavelengths and light emission intensities. Semiconductor nanoparticle fluorescent materials have excellent properties as labeling materials due to their high sensitivity, low cost, the ease of automation, and the like. Thus, a substance within particular sites of a living body or blood plasma, or the like can be rapidly detected with high sensitivity by using a semiconductor nanoparticle fluorescent material as a labeling material.

With regard to a method for detecting cancer using nanoparticles, Ivan H. El-Sayed et al., NANO LETTERS, 5, (5), 829-834 (2005) discloses a method for detecting cancer using gold nanoparticles. In this method, an anti-EGFR antibody is bound to gold nanoparticles having a diameter of approximately 35 nm, and is caused to react with an epidermal growth factor receptor (EGFR) on the surface of cancer cells (the expression level of the EGFR is low in normal cells). Cancer is detected based on the scattered light intensity or color tone that is observed to vary depending on the difference in agglomeration of the gold nanoparticles. However, such method for detecting cancer using gold nanoparticles is problematic due to its low sensitivity.

Further, Bruchez M. Jr et al., Science, 281, 2013-2015 (1998) and Chan W. C. et al., Science, 281, 2016-2018 (1998) disclose methods for detecting cancer using CdSe nanoparticles. In the methods disclosed in these documents, cancer is detected by using nanoparticle fluorescent materials containing CdSe and utilizing their property that the fluorescent materials exhibit a sharp light emission spectrum. However, these methods require complicated apparatus for detecting cancer, since CdSe is a harmful substance which makes the handling thereof cumbersome and complicated, and a sharp light emission spectrum is utilized to detect cancer.

### DISCLOSURE OF THE INVENTION

It is an object of the present invention to solve the above problems of the prior art. Namely, it is an object of the present invention to provide a nanoparticle fluorescent material for detecting cancer with which cancer can be detected with high sensitivity, using highly safe and broad light emission on a simple device. Another object of the present invention is to provide a reagent for detecting cancer and a method for detecting cancer cells using the above nanoparticle fluorescent material.

As a result of intensive studies directed towards achieving the aforementioned objects, the present inventors have found that cancer cells can be detected with high sensitivity by bringing a metal-oxide or metal-sulfide nanoparticle fluorescent material whose surface is modified with a surface modifying agent and whose half bandwidth of light emission is between 50 and 200 nm, wherein an antibody that recognizes cancer antigen is bound to the surface modifying agent, into contact with cancer cells, illuminating the material with ultraviolet light, and observing light emission therefrom. The present invention is has been completed based on such findings.

The present invention a nanoparticle fluorescent material which comprises a metal-oxide or metal-sulfide nanoparticle fluorescent material whose surface is modified by a surface modifying agent and whose half bandwidth of light emission is between 50 and 200 nm, wherein an antibody that recognizes cancer antigen is bound to the surface modifying agent.

Preferably, the surface modifying agent is a compound represented by the following formula I:
Formula I: M-(R)₄
wherein M represents Si or Ti element, and each R represents an organic group, wherein the Rs may be the same or different and at least one of the Rs represents a group having a reactivity to an antibody that recognizes cancer antigen.

Preferably, the surface modifying agent is a compound represented by the following formula II:
Formula II: HS-L-W
wherein L represents a divalent linking group, and W represents COOM or NH₂, wherein M represents a hydrogen atom, an alkali metal atom or NX₄, wherein X represents a hydrogen atom or an alkyl group.

Preferably, the nanoparticle fluorescent material is of doped-type containing 0.01 to 10 atom % of a different metal ion.

Preferably, the nanoparticle fluorescent material contains a zinc compound as a main component.

Preferably, the nanoparticle fluorescent material contains a zinc oxide as a main component.

Preferably, the antibody that recognizes cancer antigen is an anti-EGFR antibody.

Preferably, the nanoparticle fluorescent material according to the present invention is used for detecting cancer.

Another aspect of the present invention provides a reagent for detecting cancer, which comprises the nanoparticle fluorescent material according to the present invention as mentioned above.

Further another aspect of the present invention provides a dispersion liquid of a nanoparticle fluorescent material, wherein the nanoparticle fluorescent material according to the present invention as mentioned above is dispersed in either water or a hydrophilic solvent.

Further another aspect of the present invention provides a method for detecting cancer cells, which comprises the steps of: bringing the nanoparticle fluorescent material, the reagent for detecting cancer or the dispersion liquid of a nanoparticle fluorescent material according to the present invention as mentioned above into contact with a specimen, so as to allow the nanoparticle fluorescent material to bind to cancer cells; and observing light emission from the nanoparticle fluorescent material bound to the cancer cells by illuminating the specimen with excitation light.

### BRIEF DESCRIPTION OF THE DRAWINGS

The patent or application file contains at least one drawing executed in color. Copies of this patent or patent application publication with color drawings(s) will be provided by the Office upon request and payment of the necessary fees.

FIG. 1 shows the results of fluorescence microscope observation of KB cells and CHL/IU cells which were cultured by adding antibody-linked zinc oxide nanoparticles.

FIG. 2 shows the results of fluorescence microscope observation of KB cells and CHL/IU cells which were cultured by adding antibody-linked zinc sulfide nanoparticles.

### BEST MODES FOR CARRYING OUT THE INVENTION

Hereafter, embodiments of the invention will be described.

(1) Surface Modifying Agent

The surface of the metal-oxide or metal-sulfide nanoparticle fluorescent material of the present invention is modified with a surface modifying agent. The type of the surface modifying agent used in the invention is not particularly limited, as long as an antibody that recognizes cancer antigen can be bound to it. For example, the surface modifying agent may be a compound represented by the following formula I, a decomposition product thereof, a partially condensed product thereof, or a compound represented by the following formula II, for example. With such surface modifying agent, the surface of a metal-oxide or metal-sulfide nanoparticle fluorescent material can be modified. In this way, dispersibility of the nanoparticle fluorescent material in water or hydrophilic solvent can be improved, elution of the nanoparticle fluorescent material due to body fluid or the like can be prevented, and fluorescence intensity can be increased. Also, such surface modifying agent has the advantage of facilitating the binding of the antibody for detecting cancer antigens.

Hereafter, a surface modifying agent used in the invention will be described. The surface modifying agent used in the invention is a compound represented by the following formula I, a decomposition product thereof, a partially condensed product thereof, or a compound represented by the following formula II.
Formula I: M-(R)₄
wherein M represents Si or Ti element, and each R represents an organic group, wherein the Rs may be the same or different and at least one of the Rs represents a group having a reactivity to an antibody that recognizes cancer antigen.

Among the organic groups represented by R, examples of the group having a reactivity to an antibody that recognizes cancer antigen may include those to the terminal of which a vinyl group, an allyloxy group, an acryloyl group, a methacryloyl group, an isocyanato group, a formyl group, an epoxy group, a maleimide group, a mercapto group, an amino group, a carboxyl group, halogen or the like is bound via a linking group. Among such groups having reactivity, groups having an amino group at the end thereof are particularly preferable.

Examples of the linking group include alkylene groups (examples: groups having a carbon number of 1 to 10, preferably 1 to 8, and having the form of a chain or a ring, such as a methylene group, an ethylene group, a trimethylene group, a tetramethylene group, a hexamethylene group, a propylene group, an ethylethylene group, and a cyclohexylene group.)

The linking group may include an unsaturated bond. Examples of the unsaturated group include alkenylene groups (examples: groups having a carbon number of 1 to 10, preferably 1 to 8, and having the form of a chain or a ring, such as a vinylene group, a propenylene group, a 1-butenylene group, a 2-butenylene group, a 2-pentenylene group, an 8-hexadecenylene group, a 1,3-butanedienylene group, and a cyclohexenylene group), and arylene groups (for example, groups having a carbon number of 6 to 10, such as a phenylene group or a naphthylene group, preferably a phenylene group having a carbon number of 6).

The linking group may include one or more hetero atoms (which refer to any atom other than carbon atom, such as nitrogen atom, oxygen atom, or sulfur atom). Preferably, the hetero atom is oxygen atom or sulfur atom, and is most preferably oxygen atom. While the number of hetero atoms is not particularly defined, preferably it is 5 or less, and is more preferably 3 or less.

The linking group may contain, as a partial structure, a functional group containing a carbon atom adjacent to the above hetero atom. Examples of such functional group include ester groups (including carboxylic acid ester, carbonic acid ester, sulfonic acid ester, and sulfinic acid ester), amide groups (including carboxylic amide, urethane, sulfonic acid amide, and sulfinic acid amide), ether groups, thioether groups, disulfide groups, amino groups, and imido groups. The above functional group may further include a substituent, and a plurality of such functional groups may be present in each linking group. When a plurality of such functional groups are present, they may be the same or different.

A preferred functional group is an ester group, an amide group, an ether group, a thioether group, a disulfide group, or an amino group, and is more preferably an alkenyl group, an ester group, or an ether group.

While the other organic groups represented by R may be any group, preferable examples thereof include alkoxy groups and phenoxy groups such as a methoxy group, an ethoxy group, an isopropoxy group, an n-propoxy group, a t-butoxy group, or an n-butoxy group. While such alkoxy groups and phenoxy groups may further include a substituent, it is desirable that the total carbon number is 8 or less.

In the surface modifying agent represented by the formula I, a salt may be formed between the amino group or a carboxyl group and an acid or a base.

The decomposition product or the partially condensed product of the compound represented by the formula I mean a hydroxide formed by the hydrolysis of an alkoxy group, a low molecular weight oligomer (which may be a linear structure, a ring structure, a cross-linked structure or the like) produced by dehydration condensation reaction between hydroxyl groups, a product of a dealcoholization condensation reaction between a hydroxyl group and a non-hydrolyzed alkoxy group, or a sol and gel formed by dehydration condensation reaction of the above materials.

The specific examples of the surface modifying agent represented by the formula I will be described below, but the surface modifying agent used in the present invention is not limited thereto:

N-(2-aminoethyl)-3-aminopropylmethyldimethoxysilane,

N-(2-aminoethyl)-3-aminopropyltrimethoxysilane,

N-(2-aminoethyl)-3-aminopropyltriethoxysilane,

3-aminopropyltrimethoxysilane,

aminophenyltrimethoxysilane,

3-aminopropyltriethoxysilane,

bis(trimethoxysilylpropyl)amine,

N-(3-aminopropyl)-benzamidetrimethoxysilane,

3-hydrazidopropyl trimethoxysilane,

3-maleimidopropyl trimethoxysilane,

(p-carboxy)phenyltrimethoxysilane,

3-carboxypropyltrimethoxysilane,

3-aminopropyltitaniumtripropoxide,

3-aminopropylmethoxyethyltitaniumdiethoxide, and

3-carboxypropyltitaniumtrimethoxide.

In the surface modifying agent represented by the formula I, a salt may be formed between the terminal NH₂ group or COOH group and an acid or a base.

The surface modifying agent represented by the formula I may be allowed to coat the entire surface of the nanoparticle fluorescent material or bind to a part thereof. Further, in the present invention, a single or a plurality of surface modifying agents may be used.

In the present invention, in addition to the above surface modifying agent, any known surface modifying agent (such as polyethylene glycol, polyoxyethylene (1) lauryl ether phosphate, lauryl ether phosphate, trioctylphosphine, trioctylphosphine oxide, sodium polyphosphate, or bis(2-ethylhexyl)sodium sulfosuccinate) may be allowed to coexist during or after the synthesis of nanoparticles.

In the present invention, compounds represented by the following formula II may be used as the surface modifying agent.
Formula II HS-L-W
wherein L represents a divalent linking group, and W represents COOM or NH₂, wherein M represents a hydrogen atom, an alkali metal atom or NX₄, wherein X represents a hydrogen atom or an alkyl group.

Examples of the linking group includes alkylene groups (examples: groups having a carbon number 1 to 20, preferably 1 to 18, and having the form of a chain or a ring, such as a methylene group, an ethylene group, a trimethylene group, a tetramethylene group, a hexamethylene group, a propylene group, an ethylethylene group, and a cyclohexylene group).

The linking group may include an unsaturated bond. Examples of the unsaturated group include alkenylene groups (examples: groups having a carbon number of 1 to 20, preferably 1 to 18, and having the form of a chain or a ring, such as a vinylene group, a propenylene group, a 1-butenylene group, a 2-butenylene group, a 2-pentenylene group, an 8-hexadecenylene group, a 1,3-butanedienylene group, and a cyclohexenylene group), alkynylene groups (for example: groups having a carbon number of 1 to 20, preferably 1 to 18, such as an ethynylene group or a propynylene group), and arylene groups (examples: groups having a carbon number of 6 to 14, such as a phenylene group, a naphthylene group, or an anthrylene group, preferably, a phenylene group with a carbon number of 6).

The linking group may include at least one hetero atom (which refers to an any atom other than carbon atom, such as nitrogen atom, oxygen atom or sulfur atom). Preferably, the hetero atom is oxygen atom or sulfur atom, and is most preferably oxygen atom. While the number of the hetero atom is not particularly defined, preferably it is 5 or less, and is more preferably 3 or less.

The linking group may contain, as a partial structure, a functional group containing carbon atom adjacent to the above hetero atom. Examples of such functional group include an ester group (including carboxylic acid ester, carbonic acid ester, sulfonic acid ester, and sulfinic acid ester), an amide group (including carboxylic amide, urethane, sulfonic acid amide, and sulfinic acid amide), an ether group, a thioether group, a disulfide group, an amino group, and an imido group. The above functional group may further include a substituent, and a plurality of such functional groups may be present in each L. When a plurality of such functional groups are present, they may be the same or different.

A preferred functional group is an ester group, an amide group, an ether group, a thioether group, a disulfide group, or an amino group, and is more preferably an alkenyl group, an ester group, or an ether group.

If W is NH₂, it may form a salt of hydrochloric acid, sulfuric acid, nitric acid, phosphoric acid, sulfonic acid, or the like. Examples of the alkali metal atom represented by M include lithium (Li), sodium (Na), and potassium (K). Examples of the alkyl group represented by X include groups having a carbon number of 1 to 20, preferably 1 to 18, and having the form of a chain, such as a methyl group, an ethyl group, an n-propyl group, an isopropyl group, a t-butyl group, an octyl group, or a cetyl group. The four Xs may be the same or different.

Specific examples of the surface modifying agent represented by the formula II include mercaptoacetic acid, 2-mercaptopropionic acid, 3-mercaptopropionic acid, 2-mercaptobutyric acid, 4-mercaptobutyric acid, 8-mercaptooctanoic acid, 11-mercaptoundecanoic acid, 18-mercaptostearic acid, 3-mercaptoacrylic acid, mercaptomethacrylic acid, 4-mercaptocrotonic acid, 18-mercaptooleic acid, thiomalic acid, mercaptopropiolic acid, 4-mercaptophenylhydrocinnamic acid, 2-mercaptoethylamine, 2-mercaptopropylamine, 3-mercaptopropylamine, 3-mercapto-n-butylamine, 4-mercapto-n-butylamine, 2-mercapto-t-butylamine, 8-mercaptooctylamine, 11-mercaptoundecylamine, 18-mercaptostearylamine, 18-mercaptooleylamine, 5-aminopentanoic acid(2-mercapto-ethyl)-amide, 6-aminohexanoic acid(2-mercapto-ethyl)-amide, 11-aminoundecanoic acid(2-mercapto-ethyl)-amide, 5-aminopentanoic acid-3-mercapto-propyl ester, 11-amino-undecanoic acid-3-mercaptopropyl ester, 3-(11-amino-undecyloxy)-propane-1-thiol, and (2-mercapto-ethylamino)-acetic acid-2-[2-(2-aminoacetoxy)-ethoxy]-ethyl ester. Further, the compounds having an amino group may form a salt with an acid as described above. The invention is not limited to the above compounds.

With regard to the surface modifying agent used in the present invention, if water is employed for dispersion solvent, the carbon number of L is preferably between 1 and 5 and more preferably between 1 and 4. If organic solvent is employed for dispersing solvent, the carbon number of L is preferably between 6 and 20 and more preferably between 6 and 18. During or after the synthesis of nanoparticles, a known surface modifying agent (for example, polyethylene glycol, polyoxyethylene (I) lauryl ether phosphate, lauryl ether phosphate, trioctylphosphine, trioctylphosphine oxide, sodium polyphosphate, bis(2-ethylhexyl)sodium sulfosuccinate and the like) may be allowed to coexist. While the surface modifying agent represented by the formula I can be used for either metal-oxide or metal-sulfide nanoparticle fluorescent material, it is preferable that the surface modifying agent represented by the formula II is used for the metal-sulfide nanoparticle fluorescent material.

While the metal-oxide or metal-sulfide nanoparticle fluorescent material in the present invention is not particularly limited, as long as it emits fluorescence having a half bandwidth of 50 to 200 nm, examples of metal that constitutes the metal oxide or metal sulfide include metals in group 12 such as Zn, those in group 3 such as Y, Eu, and Tb, those in group 13 such as Ga and In, those in group 4 such as Zr and Hf, those in group 14 such as Si and Ge, those in group 5 such as V and Nb, and those in group 6 such as Mo and W. Among such metals, Zn is particularly preferable due to its compatibility with living bodies. Further, complex metal oxides such as Zn₂SiO₄, CaSiO₃, MgWO₄, YVO₄, and Y₂SiO₅ may be employed. Further, it is preferable that such metal-oxide or metal-sulfide nanoparticle fluorescent material contain a small quantity of metal ion which is different from the metals in the constituent metal oxide or sulfide. Examples of such metal ions include ions of metal such as Mn, Cu, Eu, Tb, Tm, Ce, Al and Ag. Preferably, such metal ion is doped as a compound in which a chloride ion or a fluoride ion is combined. The doping metal ion may consist of one or a plurality of types of atoms. A light emission wavelength varies depending on the doping metal ion. For example, if zinc sulfide (ZnS) is doped with manganese (Mn) (to be hereafter referred to as “ZnS: Mn”), the light emission is observed in orange, and if it is doped with europium (Eu), the light emission is observed in red. While the optimum concentration of the metal ion varies depending on the metal and the type thereof that constitutes the nanoparticle fluorescent material, preferably it is in the range of 0.001 to 10% by atom, and more preferably it is in the range of 0.01 to 10 atom %.

In the metal-oxide or metal-sulfide nanoparticle fluorescent material of the present invention, the half bandwidth of light emission is between 50 and 200 nm. However, in order to detect light emission with high sensitivity using a simple device, it is preferable that the half bandwidth be between 60 and 180 nm. Further, as a fluorescence labeling material, the light emission peak wavelength needs to be different from the absorption peak wavelength. Thus, in order to detect light emission with high sensitivity, it is preferable that the metal-oxide or metal-sulfide nanoparticle fluorescent material of the present invention have its light emission peak wavelength separated from its absorption peak wavelength by 20 nm or more, and more preferably by 50 nm or more. The fluorescence excitation wavelength region is 300 nm or more, and is preferably 350 nm or more in the near-ultraviolet or visible region from the viewpoint of reducing damage to the living body. Nanoparticle fluorescent materials having such light emission peak wavelength and half bandwidth can be obtained by selecting the constituent metals and the like as described above in metal-oxide or metal-sulfide nanoparticle fluorescent materials.

It is preferable that the metal-oxide or metal-sulfide nanoparticle fluorescent material of the present invention be metal-oxide nanoparticle fluorescent material that has superior coatability with the surface modifying agent used in the present invention.

The number−average particle diameter of the nanoparticle fluorescent material according to the present invention is preferably between 0.5 and 100 nm, more preferably between 0.5 and 50 nm, and even more preferably between 1 and 10 nm. The particle diameter distribution of the nanoparticle fluorescent material is preferably between 0 and 50%, more preferably between 0 and 20%, and even more preferably between 0 and 10% in terms of coefficient of variation. The coefficient of variation refers to the value obtained by dividing an arithmetic standard deviation by the number−average particle diameter and expressing the quotient in percentage (arithmetic standard deviation×100/number−average particle diameter). The particle diameter of nanoparticle fluorescent materials can be measured by a known method such as XRD or TEM.

(2) Method of Manufacturing a Nanoparticle Fluorescent Material and its Dispersion Liquid

The metal-oxide nanoparticle fluorescent material of the present invention can be obtained by various liquid phase synthesis methods such as a sol-gel method by which an organometallic compound such as alkoxide or acetylacetonato of a relevant metal is hydrolyzed; a hydroxide precipitation method by which alkali is added to an aqueous solution of a salt of a relevant metal so as to precipitate the salt as a hydroxide, followed by dehydration and annealing; an ultrasonic degradation method involving ultrasonic irradiation on the aforementioned precursor solution of a relevant metal; a microwave heating method involving microwave irradiation; a solvothermal method by which decomposition reaction is carried out at high temperature and high pressure; and spray pyrolysis involving spraying at high temperature. Alternatively, the metal-oxide nanoparticle fluorescent material of the present invention can be obtained by a gas phase synthesis method such as: a thermal CVD method or a plasma CVD method that involve organometallic compounds; a sputtering method or a laser ablation method that involve a target of a relevant metal or a relevant metal oxide.

The metal-sulfide nanoparticle fluorescent material of the present invention can be obtained by various liquid phase synthesis methods such as: a hot soap process in which a thermally decomposable metal compound, such as diethyldithiocarbamate compound of the metal, is subjected to crystal growth in a high-boiling-point organic solvent such as trialkylphosphine oxides, trialkylphosphines, or co-aminoalkanes; a coprecipitation method by which sulfide solution of sodium sulfide, ammonium sulfide, or the like is added in a solution of a salt of the metal for crystal growth; and a reverse micelle method by which the above raw material aqueous solution containing a surface active agent is allowed to be present as a reverse micelle in a non-polar organic solvent of alkanes, ethers, aromatic hydrocarbons, or the like, such that crystal growth is carried out in the reverse micelle. Further, the metal-sulfide nanoparticle fluorescent material can be obtained by the same gas phase synthesis method as in the case of the metal-oxide nanoparticle fluorescent material.

While the surface modifying agent used in the invention can be added when the nanoparticle fluorescent material is synthesized, preferably it is added after synthesis such that it is caused to bind to the nanoparticle fluorescent material and coat (surface modification) at lease part of the surface of the nanoparticles. The surface modifying agent represented by the formula I may be allowed to bind to the nanoparticle fluorescent materials in the sate that at least a portion thereof is hydrolyzed. After the nanoparticle fluorescent material is washed and purified by a conventional method such as centrifugation or filtration, it may be dispersed in a solvent containing the surface modifying agent used in the present invention (preferably, a hydrophilic organic solvent such as methanol, ethanol, isopropyl alcohol, or 2-ethoxyethanol), and may be coated.

While the added amount of the surface modifying agent used in the present invention varies depending on the particle size and particle concentration of the fluorescent material, the type (size and structure) of the surface modifying agent, and the like, preferably it is between 0.001 and 10 times mol, and more preferably it is between 0.01 and 2 times mol with respect to the metal oxide or the metal sulfide.

In the present invention, in addition to the surface modifying agent represented by the formula I or II, a known surface modifying agent may be used in combination as described above. While the added amount of such known surface modifying agent is not particularly limited, preferably it is between 0.01 and 100 times mol, and more preferably it is between 0.05 and 10 times mol. Excessive surface modifying agent can be removed through gel filtration or ultrafiltration.

With regard to the dispersion liquid of the nanoparticle fluorescent material to which the surface modifying agent is bound, since the nanoparticle concentration varies depending on the fluorescence intensity, it is not particularly limited. However, preferably it is between 0.01 mM and 1,000 mM and more preferably between 0.1 mM and 100 mM. It is preferable that the dispersion medium is the aforementioned alcohols, as well as a hydrophilic organic solvent such as DMF, DMSO or THR, or water.

Whether or not the surface of the nanoparticle fluorescent material is coated with the surface modifying agent can be determined through the recognition of regular intervals between particles when observed with a high-resolution TEM such as FE-TEM, and through a chemical analysis.

(3) Antibody that Recognizes Cancer Antigen

In the nanoparticle fluorescent material of the present invention, an antibody that recognizes cancer antigen is bound to the surface modifying agent. While the type of the cancer antigen is not particularly limited as long as it is suitable for cancer diagnosis, preferably, a free antigen can be used. Specific examples of the cancer antigen include an epidermal growth factor receptor (EGFR), an estrogen receptor (ER), and a progesterone receptor (PgR). EGFR is particularly preferable.

One of ordinary skill in the art can easily obtain the antibody that recognizes the above cancer antigens. For example, commercial items may be used, or an antibody may be prepared as needed by a known antibody preparation method using the above antigens or a partial peptide thereof as an immunogen. Also, the antibody to be used may be a monoclonal antibody or a polyclonal antibody.

The nanoparticle fluorescent material coated with the surface modifying agent can be allowed to bind to an antibody that recognizes a cancer antigen by forming a peptide bond via amidation reaction, using the amino group or carboxyl group, for example, which is a terminal group of the surface modifying agent, as a reactive group.

The amidation reaction is conducted through condensation between a carboxyl group or its derivative (ester, acid anhydride, acid halide, or the like) and an amino group. If acid anhydride or acid halide is used, it is desirable to allow a base to coexist therewith. If a carboxylic acid ester such as a methyl ester or an ethyl ester is used, it is desirable that it be heated or depressurized in order to remove alcohol that is produced. If a carboxyl group is directly amidated, a substance for promoting the amidation reaction may be allowed to coexist or to react in advance, the substance including an amidation reagent such as DCC, Morpho-CDI or WSC, a condensation additive such as HBT, an active ester agent such as N-hydroxyphthalimide, p-nitrophenyl trifluoroacetate, or 2,4,5-trichlorophenol. Further, it is desirable that, during amidation reaction, either an amino group or a carboxyl group of the affinity molecules that are to be bound through amidation be protected by an appropriate protecting group in accordance with a conventional method and be deprotected after the reaction.

After the nanoparticle fluorescent material to which an antibody that recognizes cancer antigen is bound via amidation reaction is washed and purified by a conventional method such as gel filtration, it is dispersed in water and/or hydrophilic solvent (preferably, methanol, ethanol, isopropanol, 2-ethoxyethanol, or the like) before use. The concentration of the nanoparticle fluorescent material in the dispersion liquid is not particularly limited since it varies depending on the fluorescence intensity. However, preferably the concentration is between 10⁻¹ M and 10⁻¹⁰ M, and more preferably it is between 10⁻² M and 10⁻⁶ M.

(4) Diagnosis of Cancer

The nanoparticle fluorescent material of the present invention, which is characterized in that the antibody that recognizes a cancer antigen is bound to the surface modifying agent, can be used for cancer diagnosis. Namely, the nanoparticle fluorescent material of the present invention can be used as a reagent for detecting cancer.

In the present invention, the type of the cancer to be detected is not particularly limited as long as it is a cancer in which a cancer antigen recognized by the antibody bound to the nanoparticle fluorescent material is expressed. Specific examples of such cancer include malignant melanoma, malignant lymphoma, digestive cancer, lung cancer, esophageal cancer, stomach cancer, large bowel cancer, rectal cancer, colon cancer, ureteral tumor, gallbladder cancer, cholangiocarcinoma, bile duct carcinoma, breast cancer, liver cancer, pancreatic cancer, testicular tumor, maxillary cancer, tongue cancer, lip cancer, oral cancer, pharyngeal cancer, laryngeal cancer, ovarian cancer, uterine cancer, prostatic cancer, thyroid cancer, brain tumor, Kaposi sarcoma, hemangioma, leukemia, polycythemia vera, neuroblastoma, retinoblastoma, myeloma, bladder tumor, sarcoma, osteosarcoma, myosarcoma, skin cancer, basal cell carcinoma, skin appendage carcinoma, skin metastatic carcinoma, and cutaneous melanoma. The type of cancer detected by the present invention is not limited to the above examples.

The present invention further relates to a method for detecting cancer cells with the use of the nanoparticle fluorescent material or a reagent for detecting cancer of the present invention. The detection of cancer cells can be conducted as follows. A dispersion liquid containing the nanoparticle fluorescent material of the present invention is brought into contact with a specimen such that the nanoparticle fluorescent material is allowed to bind to cancer cells. Excessive nanoparticle fluorescent material is washed and removed. Then, the specimen is irradiated with excitation light, and light emission from the nanoparticle fluorescent material bound to the cancer cells is observed.

Specifically, to a specimen containing cancer cells is added the nanoparticle fluorescent material of the present invention to which an antibody that recognizes cancer antigen is bound, to a concentration of, for example, 1.0×10⁻⁹ mol/L or higher, or generally on the order of 1.0×10⁻⁹ mol/L to 1.0×10⁻³ mol/L. Then, the cells are cultured. After the cells are washed, the cells are fixed with a conventional means and are observed with a microscope. An epifluorescent microscope may be used as a microscope. For example, fluorescence (λ max 540 nm) based on zinc oxide nanoparticles can be measured under the following conditions: light source, extra-high pressure mercury lamp; excitation filter, 330-385; excitation wavelength, 365/366; absorption filter, 420; mirror filter, 400; and ND filter, not used.

The present invention will be described in detail by the following examples, but the present invention is not limited thereto.

### EXAMPLES

### Example 1

Preparation of Dispersion Liquid of ZnO Nanoparticle Fluorescent Material

8.8 g of zinc acetate 2-hydrate was dissolved in 400 ml of dehydrated ethanol, and then 240 ml was distilled away while it was refluxed at 93° C. for 2 hours. Then, 240 ml of dehydrated ethanol was added, and the resultant was cooled to room temperature. 18 ml of methanol solution of tetramethylammonium hydroxide (25% by weight) was added and agitated for 30 minutes. 7.2 ml of 3-aminopropyltrimethoxysilane and 2.2 ml of water were added, and the mixture was agitated at 60° C. for 4 hours. Produced white deposits were filtered off and was washed with ethanol and dried. The deposits were found to be ZnO nanoparticles having an average particle diameter of about 8 nm, having Si and an aminopropyl group bound to the surface thereof, based on the analysis by XRD, TEM, elemental analysis, and IR spectrum absorption measurement method. By adding water to the deposits, water dispersion liquid (2% by weight) was prepared. The fluorescence spectrum of the dispersion liquid, when irradiated with light of 370 nm, showed broad and strong fluorescence whose peak wavelength was 540 nm and half bandwidth was 160 nm.

Binding of ZnO Nanoparticles with Anti-EGFR Antibody

An antibody Fab′ was introduced into the zinc oxide nanoparticles by a conventional hinge method (“Enzyme Immunoassay 3rd Edition”; Eiji Ishikawa et al., Igaku-Shoin Ltd.).

The nanoparticles (approximately 8 nm) comprising zinc oxide particles having an average diameter of 3 nm, whose surface was coated with aminopropylsilane, were dispersed in 0.1M HEPES (pH 8.0) buffer at a concentration of 14 mg/ml. To 1 ml of the nanoparticle dispersion liquid, 0.6 mg of Sulfo-GMBS (DOJINDO LABORATORIES) was added, allowed to react at room temperature for 1 hour, and the product was purified with PD-column (Amersham Pharmacia Bioscience), thereby obtaining a maleimide zinc oxide nanoparticle solution.

Fab′ fraction which was purified by pepsine treatment of anti-EGFR monoclonal antibodies (manufactured by Sigma Chemical Co.) and mercaptoethylamine reduction was dissolved in 0.1M HEPES (pH 7.4) buffer, and the concentration was adjusted to be 1 mg/ml. After equal volumes of the thus obtained solution and the maleimide zinc oxide nanoparticle solution (1 mg/ml 0.1M HEPES (pH 7.4)) were mixed, the mixture was agitated at 4° C. overnight. Then, the mixture was purified by gel filtration with Sephadex G100 (eluted with pH 7.4 0.1M HEPES buffer), thereby obtaining antibody-bound zinc oxide nanoparticles.

### Example 2

Preparation of Dispersion Liquid of ZnS:Mn Nanoparticle Fluorescent Material

15 g of zinc acetate 2-hydrate and 0.5 g of manganese acetate 4-hydrate were dissolved in 60 ml of water, so as to prepare solution 1. Further, 12.4 g of sodium sulfide 9-hydrate was dissolved in 60 ml of water, so as to prepare solution 2. 80 ml of water was poured into a 300 ml beaker, and while it was strongly agitated, the above solutions 1 and 2 were simultaneously added in the beaker at a rate of 10 ml/min. While white deposit was produced, it exhibited strong orange fluorescence when irradiated with ultraviolet light of 302 nm. 20 g of 2-mercaptopropionic acid was added thereto, and the mixture was agitated for 30 min. Thereafter, the mixture was allowed to stand, and the supernatant liquid was filtered with a 0.2 μm filter. The filtrate exhibited strong orange fluorescence when irradiated with ultraviolet light of 330 nm. The filtrate was ultrafiltered with a filter with a cutoff of 10000. By adding water and repeating washing/ultrafiltration, salt and excessive 2-mercaptopropionic acid were removed to purify ZnS: Mn colloidal dispersion liquid. The colloidal dispersion liquid was filtered with a 0.2 μm filter and the fluorescence spectrum of the filtrate was measured with an excitation wavelength of 326 nm. Orange light emission having a maximum in the vicinity of 580 nm and a half bandwidth of 65 nm was observed. The nanoparticle concentration in the filtrate was 16 mM. The average crystallite size of the produced ZnS: Mn, measured by XRD, was 2.8 nm.

Binding of Zinc Sulfide Nanoparticles with Anti-EGFR Antibody

Carboxylic acid on the surface of the zinc sulfide nanoparticles and an amino group of the antibody were linked via amide, so as to synthesize anti-EGFR-antibody-bound zinc sulfide nanoparticles. The nanoparticles comprising zinc sulfide particles having the average diameter 2.8 nm, whose surface was coated with 3-mercaptopropionic acid, were dispersed in 0.1M MES (pH 6.0) buffer at a concentration of 1 mg/ml. 0.2 mg of water-soluble carbodiimide (DOJINDO LABORATORIES) and 0.2 mg of hydroxysuccinimide were added to 200 μL of the nanoparticle dispersion. To the resultant mixture, 200 μL of 0.1M MES (pH 6.0) buffer containing anti-EGFR monoclonal antibody(l mg/ml) (manufactured by Sigma Chemical Co.) was added. The resultant mixture was agitated at 4° C. overnight and purified by gel filtration with Sephadex G100 (eluted with pH 7.4 0.1M HEPES buffer fluid), thereby obtaining antibody-bound zinc sulfide nanoparticles.

### Example 3

Culture of Cell Lines and Microscopic Observation

The KB tumor cell line that had been induced from cultured oral epidermal cancer of human oral epidermoid carcinoma cells (KB cells) was obtained from American Type Tissue Culture Collection. The cell line was cultured in Dulbecco's modified Eagle's Medium supplemented with 10% fetal bovine serum at 37° C. in a 5% CO₂ atmosphere for 72 hours.

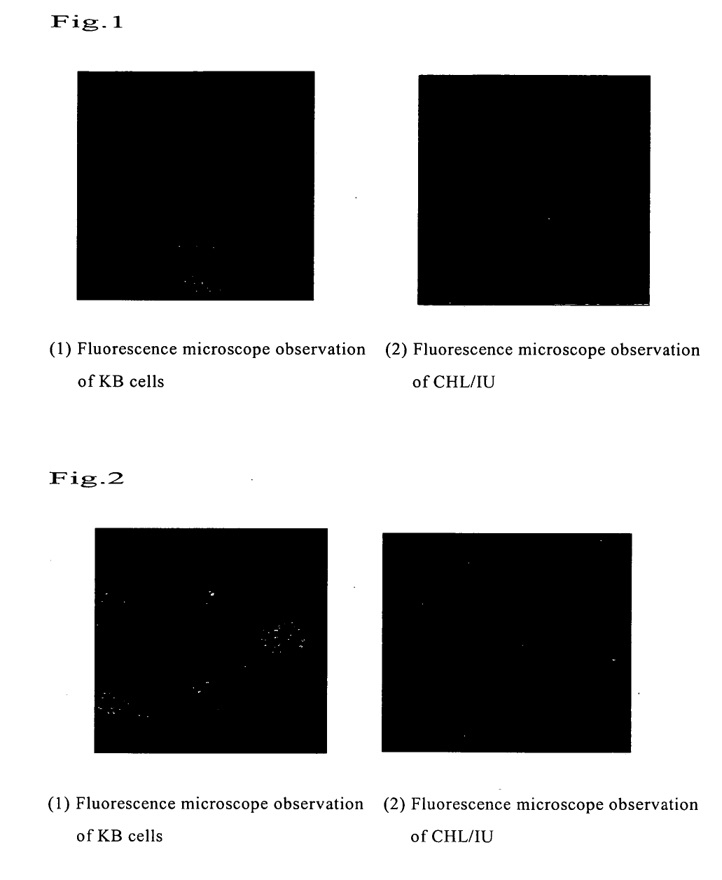

1 ml of KB (0.4×10⁴ cells/ml) was seeded on a culture slide (FALCON No. 354112) and cultured at 37° C. under 5% CO₂ for 72 hours. Then, antibody-bound-zinc oxide nanoparticles were added thereto such that the concentration was adjusted to 2.0×10⁻⁶ mol/l of the medium, and it was cultured at 37° C. under 5% CO₂ for 24 hours. The cultured cells were then washed with normal saline solution three times and fixed in 10% formalin solution at room temperature for 15 min. The resultant cells were washed with flowing water, air-dried, and observed with a microscope. As a result of observation with an epifluorescent microscope (OLYMPUS CORPORATION IX71) under the following conditions, fluorescence (λ max 540 nm) based on the zinc oxide nanoparticles accumulated on the surface of KB cells was observed ((1) in FIG. 1). Light source: extra-high pressure mercury lamp, excitation filter: 330-385, excitation wavelength: 365/366, absorption filter: 420, mirror filter: 400, and ND filter: not used.

Further, CHL cells (cultured Chinese hamster cells) were used as an example of normal cells. CHL/IU (Dainippon Pharmaceutical Co. Ltd.) was cultured with EMEM+10% BSA medium in a 35 nm petri dish at 37° C. for 72 hours. Then, 1 ml of CHL/IU (0.4×10⁴ cells/ml) was seeded on a culture slide (FALCON No. 354112) and cultured at 37° C. under 5% CO₂ for 72 hours. The fixation process was conducted under the same conditions as in the case of KB cells, followed by a fluorescence microscopic observation ((2) in FIG. 1.).

While fluorescence was clearly observed in the case of the KB cell surface, fluorescence was hardly observed in the case of CHL, presumably reflecting the difference in surface densities of EGFR that is expressed at high levels in cancer cells.

### Example 4

Culture of Cell Lines and Microscopic Observation

Similarly, KB cells and CHL/IU were observed with a fluorescence microscope using antibody-bound zinc sulfide nanoparticles. As a result, while fluorescence (λ max 580 nm) based on zinc sulfide nanoparticles was clearly observed on the KB cell surface ((1) in FIG. 2) as in the case of Example 3, only faint fluorescence was observed in the case of CHL/IU cells ((2) in FIG. 2).

### INDUSTRIALLY APPLICABILITY

In accordance with the nanoparticle fluorescent material of the invention and the method for detecting cancer using the same, cancer can be detected with high sensitivity and high safety using a simple device.

1. A nanoparticle fluorescent material which comprises a metal-oxide or metal-sulfide nanoparticle fluorescent material whose surface is modified by a surface modifying agent and whose half bandwidth of light emission is between 50 and 200 nm, wherein an antibody that recognizes cancer antigen is bound to the surface modifying agent.

2. The nanoparticle fluorescent material according to claim 1, wherein the surface modifying agent is a compound represented by the following formula I:
Formula I: M-(R)₄
wherein M represents Si or Ti element, and each R represents an organic group, wherein the Rs may be the same or different and at least one of the Rs represents a group having a reactivity to an antibody that recognizes cancer antigen.

3. The nanoparticle fluorescent material according to claim 1, wherein the surface modifying agent is a compound represented by the following formula II:
Formula II: HS-L-W
wherein L represents a divalent linking group, and W represents COOM or NH₂, wherein M represents a hydrogen atom, an alkali metal atom or NX₄, wherein X represents a hydrogen atom or an alkyl group.

4. The nanoparticle fluorescent material according to claim 1, wherein the nanoparticle fluorescent material is of doped-type containing 0.01 to 10 atom % of a different metal ion.

5. The nanoparticle fluorescent material according to claim 1, wherein the nanoparticle fluorescent material contains a zinc compound as a main component.

6. The nanoparticle fluorescent material according to claim 5, wherein the nanoparticle fluorescent material contains a zinc oxide as a main component.

7. The nanoparticle fluorescent material according to claim 1, wherein the antibody that recognizes cancer antigen is an anti-EGFR antibody.

8. The nanoparticle fluorescent material according to claim 1, which is used for detecting cancer.

9. A reagent for detecting cancer, which comprises the nanoparticle fluorescent material according to claim 1.

10. A dispersion liquid of a nanoparticle fluorescent material, wherein the nanoparticle fluorescent material according to claim 1 is dispersed in either water or a hydrophilic solvent.

11. A method for detecting cancer cells, which comprises the steps of: bringing the nanoparticle fluorescent material according to claim 1, into contact with a specimen, so as to allow the nanoparticle fluorescent material to bind to cancer cells; and observing light emission from the nanoparticle fluorescent material bound to the cancer cells by illuminating the specimen with excitation light.

12. A method for detecting cancer cells, which comprises the steps of: bringing the nanoparticle fluorescent material according to claim 2, into contact with a specimen, so as to allow the nanoparticle fluorescent material to bind to cancer cells; and observing light emission from the nanoparticle fluorescent material bound to the cancer cells by illuminating the specimen with excitation light.

13. A method for detecting cancer cells, which comprises the steps of: bringing the nanoparticle fluorescent material according to claim 3, into contact with a specimen, so as to allow the nanoparticle fluorescent material to bind to cancer cells; and observing light emission from the nanoparticle fluorescent material bound to the cancer cells by illuminating the specimen with excitation light.

14. A method for detecting cancer cells, which comprises the steps of: bringing the nanoparticle fluorescent material according to claim 4, into contact with a specimen, so as to allow the nanoparticle fluorescent material to bind to cancer cells; and observing light emission from the nanoparticle fluorescent material bound to the cancer cells by illuminating the specimen with excitation light.

15. A method for detecting cancer cells, which comprises the steps of: bringing the nanoparticle fluorescent material according to claim 5, into contact with a specimen, so as to allow the nanoparticle fluorescent material to bind to cancer cells; and observing light emission from the nanoparticle fluorescent material bound to the cancer cells by illuminating the specimen with excitation light.

16. A method for detecting cancer cells, which comprises the steps of: bringing the nanoparticle fluorescent material according to claim 6, into contact with a specimen, so as to allow the nanoparticle fluorescent material to bind to cancer cells; and observing light emission from the nanoparticle fluorescent material bound to the cancer cells by illuminating the specimen with excitation light.

17. A method for detecting cancer cells, which comprises the steps of: bringing the nanoparticle fluorescent material according to claim 7, into contact with a specimen, so as to allow the nanoparticle fluorescent material to bind to cancer cells; and observing light emission from the nanoparticle fluorescent material bound to the cancer cells by illuminating the specimen with excitation light.

18. A method for detecting cancer cells, which comprises the steps of: bringing the nanoparticle fluorescent material according to claim 8, into contact with a specimen, so as to allow the nanoparticle fluorescent material to bind to cancer cells; and observing light emission from the nanoparticle fluorescent material bound to the cancer cells by illuminating the specimen with excitation light.

19. A method for detecting cancer cells, which comprises the steps of: bringing the reagent for detecting cancer according to claim 9, into contact with a specimen, so as to allow the nanoparticle fluorescent material to bind to cancer cells; and observing light emission from the nanoparticle fluorescent material bound to the cancer cells by illuminating the specimen with excitation light.

20. A method for detecting cancer cells, which comprises the steps of: bringing the dispersion liquid of a nanoparticle fluorescent material according to claim 10 into contact with a specimen, so as to allow the nanoparticle fluorescent material to bind to cancer cells; and observing light emission from the nanoparticle fluorescent material bound to the cancer cells by illuminating the specimen with excitation light.
